(19) United States
(12) Patent Application Publication
(10) Pub. No.: US 2022/0296559 A1
(43) Pub. Date: Sep. 22, 2022

(54) Composition of a Spray-Dried Powder for Pulmonary Delivery of a Long Acting Neuraminidase Inhibitor (LANI)

(71) Applicant: Civitas Therapeutics, Inc., Ardsley, NY (US)

(72) Inventors: Kevin L. Ward, Arlington, MA (US); Thean Y. Yeoh, Foxboro, MA (US); Rebecca Martin, Blacksburg, VA (US); Charles D. Blizzard, Westwood, MA (US)

(21) Appl. No.: 17/517,157

(22) Filed: Nov. 2, 2021

Related U.S. Application Data
(63) Continuation of application No. 17/215,554, filed on Mar. 29, 2021, now abandoned.
Application No. 17/215,554 is a continuation of application No. 17/001,794, filed on Aug. 25, 2020, now abandoned.
Application No. 17/001,794 is a continuation of application No. 16/722,656, filed on Dec. 20, 2019, now abandoned.
Application No. 16/722,656 is a continuation of application No. 16/408,554, filed on May 10, 2019, now abandoned.
Application No. 16/408,554 is a continuation of application No. 16/127,648, filed on Sep. 11, 2018, now abandoned.
Application No. 16/127,648 is a continuation of application No. 15/864,372, filed on Jan. 8, 2018.
Application No. 15/864,372 is a continuation of application No. 15/213,886, filed on Jul. 19, 2016, now abandoned.
Application No. 15/213,886 is a continuation of application No. 11/838,468, filed on Aug. 14, 2007, now abandoned.
(60) Provisional application No. 60/843,320, filed on Sep. 8, 2006.

(51) Int. Cl. A61K 31/351; A61K 9/00; A61K 9/16; A61K 31/196

(52) U.S. Cl. CPC A61K 31/351; A61K 9/0075; A61K 9/1611; A61K 9/1617; A61K 31/196

(57) ABSTRACT

The present invention is related to pharmaceutical formulations and methods of treating a subject afflicted with the influenza virus, the method includes administering to the respiratory tract of the patient particles that include more than about 5% to about 50% weight percent (wt %) of a neuraminidase inhibitor. The particles are delivered to the patient's pulmonary system, including the upper airways, central airways and deep lung.

### RELATED APPLICATIONS

This application is a continuation of U.S. application Ser. No. 17/215,554, filed Mar. 29, 2021, which is a continuation of U.S. application Ser. No. 17/001,794, filed Aug. 25, 2020, now abandoned, which is a continuation of U.S. application Ser. No. 16/722,656, filed Dec. 20, 2019, now abandoned, which is a continuation of U.S. application Ser. No. 16/408,554, filed May 10, 2019, now abandoned, which is a continuation of U.S. application Ser. No. 16/127,648, filed Sep. 11, 2018, now abandoned, which is a continuation of U.S. application Ser. No. 15/864,372, filed Jan. 8, 2018, now abandoned, which is a continuation of U.S. application Ser. No. 15/213,886, filed Jul. 19, 2016, now abandoned, which is a continuation of U.S. application Ser. No. 11/838,468, filed Aug. 14, 2007, now abandoned, which claims the benefit of U.S. Provisional Application No. 60/843,320, filed on Sep. 8, 2006. The entire teachings of the above applications are incorporated herein by reference.

### TECHNICAL FIELD

The present invention relates to pharmaceutical formulations comprising a neuraminidase inhibitor for the treatment of influenza types A and B by pulmonary administration to a subject in need of treatment.

### BACKGROUND OF THE INVENTION

Influenza viruses are divided into three types, designated A, B, and C. Influenza types A or B cause epidemics of disease almost every winter. In the United States alone, types A and B can cause illness in 10% to 20% of people and are associated with an average of 36,000 deaths and 114,000 hospitalizations per year (Centers for Disease Control and Prevention (CDC)). Influenza types A and B are particularly dangerous for young children, elderly individuals, and for chronically ill patients. Common symptoms associated with types A and B generally include fever from about 100° C.-104° C., shaking chills, body aches, headaches, fatigue, cough, and sore throat. In contrast, influenza type C differs from types A and B in some important ways. Type C infection usually causes either a mild respiratory illness or no symptoms at all; it does not cause epidemics and does not have the severe public health impact that influenza types A and B do.

Influenza type A viruses are divided into subtypes based on two proteins on the surface of the virus: the hemagglutinin (H) and the neuraminidase (N). The current subtypes of influenza A viruses found in people are A(H1N1) and A(H3N2). Influenza A viruses are also found in many animals, including ducks, chickens, wild birds, pigs, whales, horses, and seals. Influenza viruses tend to be species specific, however, sporadic human infections and outbreaks caused by certain avian influenza A viruses have been reported (Li, K.S. et al., 2004). In contrast, influenza type B virus is not divided into subtypes and circulate widely only among humans.

Influenza viruses continually change over time, usually by mutation. This constant changing enables the virus to evade the immune system of its host, so that individuals are susceptible to influenza virus infections throughout their lifetime. The virus can further rearrange its RNA by mixing with other influenza viruses to create hybrid viruses that have new “H” and “N” antigens in the same virus. This occurs when an influenza virus from two different species infect the same cell. For example, the viruses could reassort and produce a new virus that had most of the genes from the human virus, but a hemagglutinin and/or neuraminidase from the avian virus. The resulting new virus would likely be able to infect humans and spread from person to person, but it would have surface proteins (hemagglutinin and/or neuraminidase) not previously seen in influenza viruses that infect humans. This type of major change in the influenza A virus is known as antigenic shift. Antigenic shift results when a new influenza A subtype to which most people have little or no immune protection infects humans. If this new virus causes illness in people and can be transmitted easily from person to person, an influenza pandemic can occur.

Influenza antiviral medications have long been used to limit the spread and impact of influenza outbreaks. In the United States, four antiviral medications (amantadine (SYMMETREL®), rimantadine (FLUMADINE®), oseltamivir (TAMIFLU®), and zanamivir (RELENZA®)) are approved for treatment of influenza A viruses. Earlier research has shown that all four antiviral medications were similarly effective in reducing the duration by 1 or 2 days of illness caused by influenza A viruses, when used for treatment within the first 2 days of illness. However, recent evidence indicates that a high proportion of currently circulating influenza A viruses in the United States have developed resistance to amantadine and rimantadine. Oseltamivir and zanamivir are taught to be effective against influenza B viruses.

Therefore, a need exists for pharmaceutical formulations and methods of treating subjects suffering with an influenza type A and B viral infection, which are at least as effective as conventional therapies and is also effective against treating virus strains resulting from mutations or resortment of the influenza virus.

### SUMMARY OF THE INVENTION

The invention relates to pharmaceutical formulations and methods of treating a subject afflicted with an influenza type A or B viral infection. Suitable neuraminidase inhibitors for use in any of the methods of the invention include, but are not limited to, CS-8958 (R118958; Sankyo Co.), zanamivir (GG167, RELENZA®; GlaxoSmithKline), peramivir (RWJ-270201, BCX-1812; BioCryst), oseltamivir phosphate (Ro64-0796, GS4104; ROCHE PHARMA®), oseltamivir carboxylate (Ro64-0802, GS4071; ROCHE PHARMA®), oseltamivir (GS4104, TAMIFLU®; ROCHE PHARMA®). The pharmaceutical formulation of the present invention includes particles comprising a neuraminidase inhibitor, preferably, a long acting neuraminidase inhibitor i.e., CS-8958 (Sankyo Co.). The method includes administering to the respiratory tract of a subject in need of treatment particles comprising an effective amount of the neuraminidase inhibitor effective to ameliorate or alleviate at least one symptom of an influenza type A or B viral infection. The particles are delivered to the pulmonary system e.g., deep lung, central airways or upper airways and the medicament is released into the patient's blood stream to reach the medicament's site of action.

The current invention provides a pharmaceutical formulation for the treatment an influenza type A or B viral infection comprising a mass of biocompatible particles that comprise, by weight, about 5% to about 50% of a neuraminidase inhibitor, preferably, CS-8958 (Sankyo Co.), a salt, preferably sodium chloride, and a material selected from the group consisting of a buffer, preferably sodium phosphate, an amino acid, preferably, leucine, and any combination thereof, wherein the particles are delivered to the pulmonary system.

In one aspect, the mass of biocompatible particles comprise a mass from about 1 mg to 20 mg of a neuraminidase inhibitor, preferably, CS-8958 (Sankyo Co.). In another aspect, the particles have a tap density of less than about 0.4 g/cm³, preferably less than about 0.1 g/cm³. In yet another aspect, the particles have a fine particle fraction of less than 5.8 of at least 45% by weight. In still another aspect, the particles have a median geometric diameter of from about 5 micrometers to about 30 micrometers, preferably from about 6 to about 8 micrometers. In yet another aspect, the particles have an aerodynamic diameter from about 1 micrometer to about 5 micrometers, preferably, from about 1 micrometer to about 3 micrometers.

The invention also relates to a pharmaceutical formulation having particles comprising, by weight, about 5% to about 30% of a neuraminidase inhibitor, preferably, CS-8958 (Sankyo Co.), about 5% to about 20% sodium chloride, about 20% to about 85% leucine and about 5% to about 20% sodium phosphate.

In another embodiment, the invention relates to a pharmaceutical formulation having particles comprising of 30% of a neuraminidase inhibitor, preferably, CS-8958 (Sankyo Co.), 15% sodium chloride, 50% leucine and 5% sodium phosphate.

In yet another embodiment, the invention relates to a pharmaceutical formulation having particles comprising of 5% of a neuraminidase inhibitor, preferably, CS-8958 (Sankyo Co.), 5% sodium chloride, 85% leucine and 5% sodium phosphate.

The invention further relates to a method of treating a human subject in need of a neuraminidase inhibitor, preferably, CS-8958 (Sankyo Co.), comprising administering pulmonarily to the respiratory tract e.g., deep lung, central airways and/or upper airways of a subject in need of treatment e.g., influenza, an effective amount of particles comprising by weight, about 5% to about 30% of a neuraminidase inhibitor, about 5% to about 20% sodium chloride, about 20% to about 85% leucine and about 5% to about 20% sodium phosphate, wherein the release of the neuraminidase inhibitor is rapid.

In one embodiment, the invention relates to a method of treating a human subject in need of a neuraminidase inhibitor, preferably, CS-8958 (Sankyo Co.), comprising administering pulmonarily to the respiratory tract of a subject in need of treatment an effective amount of particles comprising, by weight, 30% of a neuraminidase inhibitor, 15% sodium chloride, 50% leucine and 5% sodium phosphate, wherein the release of the neuraminidase inhibitor is rapid.

In another embodiment, the invention relates to a method of treating a human subject in need of a neuraminidase inhibitor, preferably, CS-8958 (Sankyo Co.), comprising administering pulmonarily to the respiratory tract of a subject in need of treatment an effective amount of particles comprising, by weight, 5% of a neuraminidase inhibitor, 5% sodium chloride, 85% leucine and 5% sodium phosphate, wherein the release of the neuraminidase inhibitor is rapid.

This invention also relates to a method of treating a subject with influenza, comprising: administering to the respiratory tract of the patient an effective amount of particles comprising by weight, about 5% to about 30% of a neuraminidase inhibitor, preferably, CS-8958 (Sankyo Co.), about 5% to about 20% sodium chloride, about 20% to about 85% leucine and about 5% to about 20% sodium phosphate, wherein the particles are delivered to the pulmonary system.

This invention further relates to a method of delivering an effective amount of a neuraminidase inhibitor to the pulmonary system, comprising: providing a mass of particles comprising, by weight, about 5% to about 30% of a neuraminidase inhibitor, preferably, CS-8958 (Sankyo Co.), about 5% to about 20% sodium chloride, about 20% to about 85% leucine and about 5% to about 20% sodium phosphate.

The invention still further relates to a pharmaceutical kit for administration of a neuraminidase inhibitor, preferably, CS-8958 (Sankyo Co.), comprising at least one receptacle, wherein said receptacle comprise unit dosages of particles comprising, by weight, about 5% about 30% of a neuraminidase inhibitor, about 5% to about 20% sodium chloride, about 20% to about 85% leucine and about 5% to about 20% sodium phosphate.

In one aspect, the kit further comprises instructions for use of said at least one receptacle.

This invention also relates to method of producing spray dried particles suitable for inhalation that comprises: a) combining a neuraminidase inhibitor, preferably, CS-8958 (Sankyo Co.), a salt, an amino acid, a buffer and co-solvent, said co-solvent including an aqueous solvent and an organic solvent e.g., ethanol to form a mixture; and (b) spray-drying said mixture to produce spray-dried particles and wherein the neuraminidase inhibitor is present in the particles in an amount of at least about 5% by weight.

### BRIEF DESCRIPTION OF THE DRAWINGS

FIG. 1. Physical stability testing by short term humidity exposure.

FIG. 2. Spray-dried powders containing leucine, sodium chloride, and sodium phosphate with 5%-40% CS-8958.

### DETAILED DESCRIPTION OF THE INVENTION

The invention is generally related to pharmaceutical formulations and methods of treating influenza types A and B viral infections. The pharmaceutical formulation includes particles comprising a neuraminidase inhibitor, preferably, a long acting neuraminidase inhibitor i.e., CS-8958 (Sankyo Co.). The method includes administering to the respiratory tract of a patient in need of treatment particles comprising an effective amount of a neuraminidase inhibitor to ameliorate or alleviate at least one symptom associated with an influenza type A or B viral infection. The particles are delivered to the pulmonary system e.g., deep lung, central airways or upper airways wherein the medicament is released into the patient's blood stream to reach the medicament's site of action.

Influenza types A and B are typically associated with influenza outbreaks in human populations. However, type A influenza also infects other creatures as well, e.g., birds, pigs, and other animals. The type A viruses are categorized into subtypes based upon differences within their hemagglutinin and neuraminidase surface glycoprotein antigens. Hemagglutinin in type A viruses have 14 known subtypes and neuraminidase has 9 known subtypes. In humans, currently only about 3 different hemagglutinin and 2 different neuraminidase subtypes are known, e.g., H1, H2, H3, N1, and N2. In particular, two major subtypes of influenza A have been active in humans, namely, H1N1 and H3N2. Influenza B viruses are not divided into subtypes based upon their hemagglutinin and neuraminidase proteins.

In order that the present invention may be more readily understood, certain terms are first defined. Additional definitions are set forth throughout the detailed description.

The term “influenza virus” as is used here to refer to any strain of influenza virus that is capable of causing disease in an animal or human subject. Influenza viruses are described in Fields, B., et al., Fields' Virology, 4^{th} Edition, Philadelphia: Lippincott Williams and Wilkins; ISBN: 0781718325, 2001. In particular, the term encompasses any strain of influenza type A virus that is capable of causing disease in an animal or human subject. A large number of influenza type A isolates have been partially or completely sequenced. A list of complete sequences for influenza A genome segments that have been deposited in a public database can be found at: (The Influenza Sequence Database (ISD), see Macken, C., Lu, H., Goodman, J., & Boykin, L., “The value of a database in surveillance and vaccine selection.” in Options for the Control of Influenza IV. A. D. M. E. Osterhaus, N. Cox & A. W. Hampson (Eds.) Amsterdam: Elsevier Science, 2001, 103-106). This database also contains complete sequences for influenza B and C genome segments. Influenza sequences are also available on Genbank. Sequences of influenza genes are therefore readily available to, or determinable by, those of ordinary skill in the art.

The term “subject” as used herein refers to any animal having a disease or condition which requires treatment with a pharmaceutically active agent e.g., a neuraminidase inhibitor, preferably, CS-8958 (Sankyo Co). The subject may be a mammal, preferably a human, or may be a non-human primate or non-primates such as used in animal model testing.

Various aspects of the invention are described in further detail in the following subsections.

### Compositions and Pharmaceutical Formulations

Neuraminidase is an essential enzyme for the replication of the influenza virus and it has been described as “molecular scissors” which cut the nascent viruses free. More specifically, the neuraminidase enzyme cleaves terminal neuraminic (sialic) acid residues from carbohydrate moieties on host epithelial cell membrane proteins, and on viral envelope glycoprotein spikes of newly synthesized virions. Generally speaking, neuraminidase enables the release of influenza virions from infected cells, promotes the dissemination of virus within the respiratory tract, and may also reduce the ability of respiratory mucus to inactivate the virus. Inhibition of the neuraminidase enzyme promotes the aggregation of viral particles on the surface of infected cells and effectively interrupts the replicative cycle of the virus.

Neuraminidase inhibitors include analogues of sialic acid, which represent a new class of second-generation anti-viral agents that show efficacy against both influenza type A and B viruses. These agents interact with a common region of the active site located in a central cleft that is conserved among all type A and type B influenza viruses studied to date despite wide variation in other regions of the enzyme. Neuraminidase inhibitors have been referred to as “plug drugs” and their proposed mechanism of action is to block the active site of the neuraminidase enzyme which effectively leaves uncleaved sialic acid residues on the surface of the host cells and viral envelopes. In the presence of a neuraminidase inhibitor, viral hemagglutinin binds to the uncleaved sialic residues, resulting in viral aggregation at the host cell surface. This inhibition of viral budding results in the overall reduction of the amount of virus that is released from infected cells.

As used herein, the term “neuraminidase inhibitor” includes agents capable of inhibiting at least one enzymatic activity that typifies a neuraminidase protein obtained from a virulent strain of a type A or type B influenza virion for a time sufficient to confer either a prophylactic or therapeutic benefit to the subject to whom it is administered. The prophylactic and treatment protocols of the invention contemplate administration of particles comprising an effective amount of a neuraminidase inhibitor to alleviate or ameliorate at least one symptom associated with the effects of an influenza type A and B viral infection. Among the numerous neuraminidase inhibitors taught by the prior art are those compounds described by Luo et al., in U.S. Pat. No. 5,453,533, by Bischofberger et al., in U.S. Pat. No. 5,763,483, by Bischofberger et al., in U.S. Pat. No. 5,952,375, by Bischofberger et al., in U.S. Pat. No. 5,958,973, by Kim et al., in U.S. Pat. No. 5,512,596, by Kent et al., in U.S. Pat. No. 5,886,213, by Babu et al., in U.S. Pat. No. 5,602,277, by Babu et al., in U.S. 6,410,594, by von Izstein et al., in U.S. Pat. No. 5,360,817, by Lew et al., in U.S. Pat. No. 5,866,601, by Brouillette et al., in U.S. Pat. No. 6,509,359, by Maring et al., in U.S. Pat. No. 6,831,096, by Maring et al., in U.S. Pat. No. 6,593,314, by Maring et al., in U.S. Pat. No. 6,518,305, and by Maring et al., in U.S. Pat. No. 6,455,571. The various neuraminidase inhibitors taught by these enumerated patents are incorporated herein by reference.

Suitable neuraminidase inhibitors for use in any of the methods of the present invention also include, but are not limited to, CS-8958 (R118958; Sankyo Co.), zanamivir (GG167, RELENZA®; GlaxoSmithKline), peramivir (RWJ-270201, BCX-1812; BioCryst), oseltamivir phosphate (Ro64-0796, GS4104; ROCHE PHARMA®), oseltamivir carboxylate (Ro64-0802, GS4071; ROCHE PHARMA®), oseltamivir (GS4104, TAMIFLU®; ROCHE PHARMA®). CS-8958 can be prepared according to the methods described in U.S. Pat. No. 6,340,702 to Honda et al., U.S. Pat. No. 6,451,766 to Honda et al., and U.S. application Ser. No. 09/969,851 filed on Oct. 3, 2001 to Honda et al., the disclosures of which are hereby incorporated by reference. Oseltamivir can be prepared according to the methods described in U.S. Pat. No. 5,763,483 to Bischofberger et al., and U.S. Pat. No. 5,866,601 to Lew et al., the disclosures of which are hereby incorporated by reference. Zanamivir can be prepared and according to the methods described in U.S. Pat. Nos. 6,294,572, 5,648,379, and 5,360,817, the disclosures of which are hereby incorporated by reference. Peramivir can be prepared according to the methods described in U.S. Pat. No. 6,503,745, the disclosures of which are hereby incorporated by reference. Whenever a neuraminidase inhibitor is mentioned herein, all of its chemical forms are included, e.g., enantiomer, diastereomer, salt, racemic, optically pure, and/or salt-free form.

Preferred compounds of the present invention used for treating an influenza type A and

B viral infection is an ester prodrug of a neuraminidase inhibitor, preferably CS-8958 (Sankyo Co.) as shown below:

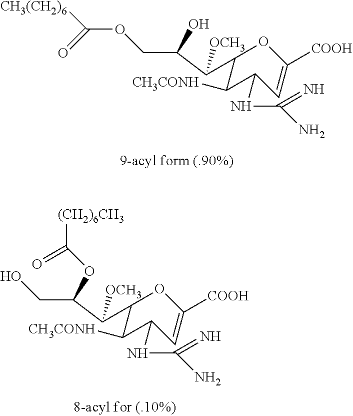

In one embodiment of the invention the biocompatible particles include a neuraminidase inhibitor, preferably, CS-8958 (Sankyo Co.) as described above. Particularly preferred are particles that include more than about 5% weight percent (wt. %), for instance, at least 5%-50% weight percent of a neuraminidase inhibitor. In one embodiment, the particles include at least 5%, 10%, 15%, 20%, 25%, 30%, 35%, 40%, 45%, 50%, 55%, 60%, 65%, 70%, 75%, or 80% wt. % of the neuraminidase inhibitor. In other embodiments, the presence of an amino acid, buffer or a salt, as will be described herein, facilitates a lower neuraminidase inhibitor weight percentage while maintaining favorable features e.g., stability of the neuraminidase inhibitor and formulation.

Without wishing to be held to a particular interpretation of the invention, it is believed that the amino acid is useful as a bulking agent, due to its low hygroscopicity and crystalline nature. This characteristic often results in powders with improved physical stability and dispersability.

Examples of amino acids which can be employed include, but are not limited to, glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine and glycine. Combinations of hydrophobic amino acids can also be employed. Furthermore, combinations of hydrophobic and hydrophilic (preferentially partitioning in water) amino acids, where the overall combination is hydrophobic, can also be employed.

The amino acid, preferably leucine, is present in the biocompatible particles of the invention in an amount of at least 20 weight percent (wt. %). Preferably, the amino acid is present in the particles in an amount ranging from about 20% to about 85 wt. %. In one embodiment, the amino acid is present in an amount of at least 10%, 15%, 20%, 25%, 30%, 35%, 40%, 45%, 50%, 55%, 60%, 65%, 70%, 75%, 80%, 85%, or 90% wt. %.

Without wishing to be held to a particular interpretation of the invention, it is believed that a buffer, such as sodium phosphate, reduces the tendency of pH of the composition to change over time as would otherwise occur due to chemical reactions. Preferably, the pH can range from about 3 to about 10. In a more preferred embodiment, the powders were prepared from solutions containing a pH of 7.

Examples of buffers which can be employed include, but are not limited to: sodium phosphate, sodium acetate, sodium carbonate, citrate, glycylglycine, histidine. lysine, arginin, TRIS, glycine and sodium citrate or mixtures thereof.

The buffer, preferably sodium phosphate, is present in the biocompatible particles of the invention in an amount of at least 5 weight percent (wt. %). Preferably, the buffer is present in the particles in an amount ranging from about 5% to about 20 wt. %. In one embodiment, the buffer is present in an amount of at least 5%, 10%, 15%, 20%, 25%, 30%, 35%, 40%, 45%, or 50% wt. %.

Without wishing to be held to a particular interpretation of the invention, it is believed that the salt, such as sodium chloride, provides a source of mobile counter-ions. It is believed that the addition of a small salt to particles that have local areas of charge on their surface will reduce the amount of static present in the final powder by providing a source of mobile counter-ions that would associate with the charged regions on the surface. Thereby the yield of the powder produced is improved by reducing powder agglomeration, improving the Fine Particle Fraction (FPF) and emitted dose of the particles and allowing for a larger mass of particles to be packed into a single receptacle.

Examples of salts which can be employed include, but are not limited to: sodium chloride, sodium phosphate, sodium fluoride, sodium sulfate and calcium carbonate.

The salt, preferably sodium chloride, is present in the biocompatible particles of the invention in an amount of at least 5 weight percent (wt. %). Preferably, the salt is present in the particles in an amount ranging from about 5% to about 20 wt. %. In one embodiment, the amino acid is present in an amount of at least 5%, 10%, 15%, 20%, 25%, 30%, 35%, 40%, 45%, or 50% wt. %.

A preferred composition consists essentially of a pharmaceutical formulation having about 5% to about 30% of a neuraminidase inhibitor, about 5% to about 20% sodium chloride, about 20% to about 85% leucine and about 5% to about 20% sodium phosphate.

In further embodiments, the particles of the invention can optionally include one or more additional component(s), e.g., phospholipids, also referred to herein as phosphoglyceride or a non-reducing sugar in combination with or without the excipients as described above.

In a preferred embodiment, the phospholipid, is endogenous to the lung. Such a phospholipid is particularly advantageous in preparing spray-dried particles suitable for delivery to the respiratory system of a patient. In another preferred embodiment the phospholipid includes, among others, phosphatidylcholines, phosphatidylethanolamines, phosphatidylglycerols, phosphatidylserines, phosphatidylinositols and combinations thereof. Specific examples of phospholipids include but are not limited to phosphatidylcholines dipalmitoyl phosphatidylcholine (DPPC), dipalmitoyl phosphatidylethanolamine (DPPE), distearoyl phosphatidylcholine (DSPC), dipalmitoyl phosphatidyl glycerol (DPPG) or any combination thereof.

Examples of non-reducing sugars which can be employed include, but are not limited to, mannitol, trehalose, sucrose, sorbitol, fructose, maltose, lactose or dextrans or any combination thereof.

### Methods Treatment and Administration

The method of the invention includes delivering to the pulmonary system an effective amount of a medicament such as, for example, neuraminidase inhibitor, preferably, CS-8958 (Sankyo Co.). As used herein, the term “effective amount” is meant an amount of the neuraminidase inhibitor, preferably, CS-8958 (Sankyo Co.) effective to prevent or treat an influenza type A and B viral infection in order to yield a desired therapeutic response. For example, an amount of a neuraminidase inhibitor capable of ameliorating or alleviating the effects of an influenza type A and B viral infection. The actual effective amounts of drug can vary according to the specific drug or combination thereof being utilized, the particular composition formulated, the mode of administration, and the age, weight, condition of the patient, and severity of the episode being treated. Dosages for a particular patient are described herein and can be determined by one of ordinary skill in the art using conventional considerations, (e.g., by means of an appropriate, conventional pharmacological protocol). For example, effective amounts of the neuraminidase inhibitor, preferably, CS-8958 (Sankyo Co.), range from about 1 milligrams (mg) to about 100 mg. In another embodiment, at least 1 milligram of a neuraminidase inhibitor, preferably, CS-8958 (Sankyo Co.), is delivered by administering, in a single breath, to a subject's respiratory tract the biocompatible particles enclosed in the receptacle. Preferably at least 10 milligrams of neuraminidase inhibitor, preferably, CS-8958 (Sankyo Co.), is delivered to a subject's respiratory tract. Amounts as high as 15, 20, 25, 30, 35, 40 and 50 milligrams can be delivered.

The terms “treating”, “treatment” and the like are used herein to mean affecting a subject, tissue or cell to obtain a desired pharmacologic and/or physiologic effect. The effect may be prophylactic in terms of completely or partially preventing an influenza type A and B viral infection or sign or symptom thereof, and/or may be therapeutic in terms of a partial or complete cure of an influenza type A and B viral infection. Symptoms associated with an influenza type A or B viral infection include, but are not limited to: fever from about 100° C.-104° C., shaking chills, body aches, headaches, fatigue, cough, and sore throat.

The invention is also related to methods for administering to the pulmonary system a therapeutic dose of the medicament in a small number of steps, and preferably in a single, breath activated step. The invention also is related to methods of delivering a therapeutic dose of a drug, preferably, CS-8958 (Sankyo Co.), to the pulmonary system, in a small number of breaths, and preferably in one or two single breaths. As used herein the term “therapeutically-effective amount” means an amount of a neuraminidase inhibitor, preferably, CS-8958 (Sankyo Co.) to yield a desired therapeutic response. For example, treating or preventing an influenza type A or B viral infection. The specific “therapeutically-effective amount” will, obviously, vary with such factors as the particular influenza viral infection being treated, the physical condition of the subject, the duration of the treatment, the nature of concurrent therapy (if any), and the specific formulation employed and the structure of the compound or its derivatives. The methods include administering the biocompatible particles from a receptacle having, holding, containing, storing or enclosing a mass of particles, to a subject's respiratory tract.

In one embodiment, at least 80% of the mass of the biocompatible particles stored in the inhaler receptacle is delivered to a subject's respiratory system in a single, breath-activated step. As used herein, the term “receptacle” includes but is not limited to, for example, a capsule, blister, film covered container well, chamber and other suitable means of storing a powder in an inhalation device known to those skilled in the art.

In a preferred embodiment, the receptacle is used in a dry powder inhaler. Examples of dry powder inhalers that can be employed in the methods of the invention include but are not limited to the inhalers disclosed is U.S. Pat. Nos. 4,995,385 and 4,069,819, the SPINHALER®. (Fisons, Loughborough, U.K.), ROTAHALER®. (Glaxo-Wellcome, Research Triangle Technology Park, North Carolina), FLOWCAPS®. (Hovione, Loures, Portugal), INHALATOR®. (Boehringer-Ingelheim, Germany), and the AEROLIZER®. (Novartis, Switzerland), the Diskhaler (Glaxo-Wellcome, RTP, NC) and others known to those skilled in the art.

In one embodiment, the volume of the receptacle is at least about 0.37 cm³. In another embodiment, the volume of the receptacle is at least about 0.48 cm cm³. In yet another embodiment, are receptacles having a volume of at least about 0.67 cm cm³ or 0.95 cm cm³. In one embodiment of the invention, the receptacle is a capsule designated with a capsule size 2, 1, 0, 00 or 000. Suitable capsules can be obtained, for example, from Shionogi (Rockville, Md.). Blisters can be obtained, for example, from Hueck Foils, (Wall, N.J.).

The receptacle encloses or stores particles, also referred to herein as powders. The receptacle is filled with particles, as known in the art. For example, vacuum filling or tamping technologies may be used. Generally, filling the receptacle with powder can be carried out by methods known in the art. In one embodiment of the invention, the article or powder enclosed or stored in the receptacle have a mass of at least about 1 milligram to at least about 20 milligrams. In one embodiment, the powder enclosed or stored in the receptacle is present in an amount of at least 1, 3, 5, 7, 10, 13, 15, 17, 20, 23, 25, 27, or 30 milligrams.

Delivery to the pulmonary system of particles in a single, breath-actuated step is enhanced by employing particles which are dispersed at relatively low energies, such as, for example, at energies typically supplied by a subject's inhalation. Such energies are referred to herein as “low.” As used herein, “low energy administration” refers to administration wherein the energy applied to disperse and/or inhale the particles is in the range typically supplied by a subject during inhaling.

The invention is also related to methods for efficiently delivering powder particles to the pulmonary system. For example, but not limited to, at least about 70% or at least about 80% of the nominal powder dose is actually delivered. As used herein, the term “nominal powder dose” is the total amount of powder held in a receptacle, such as employed in an inhalation device. As used herein, the term nominal drug dose is the total amount of medicament contained in the nominal amount of powder. The nominal powder dose is related to the nominal drug dose by the load percent of drug in the powder.

Properties of the particles enable delivery to patients with highly compromised lungs where other particles prove ineffective for those lacking the capacity to strongly inhale, such as young patients, old patients, infirm patients, or patients with asthma or other breathing difficulties. Further, patients suffering from a combination of ailments may simply lack the ability to sufficiently inhale. Thus, using the methods and particles for the invention, even a weak inhalation is sufficient to deliver the desired dose.

### Administration of Biocompatible Particles

Particles of the invention are suitable for delivering a neuraminidase inhibitor, preferably, CS-8958 (Sankyo Co.) to the pulmonary system. Particles suitable for use in the methods of the invention can travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. In one embodiment of the invention, most of the mass of particles deposit in the deep lung or alveoli. In another embodiment of the invention, delivery is primarily to the central airways. In other embodiments, delivery is to the upper airways.

The particles of the invention can be administered as part of a pharmaceutical formulation or in combination with other therapies be they oral, pulmonary, by injection or other mode of administration. As described herein, particularly useful pulmonary formulations are spray dried particles having physical characteristics characterized by a fine particle fraction (FPF), geometric and aerodynamic dimensions and by other properties which favor target lung deposition and are formulated to optimize release and bioavailability profiles, as further described below.

Gravimetric analysis, using Cascade impactors, is one method of measuring the size distribution of airborne particles. The Andersen Cascade Impactor (ACI) is an eight-stage impactor that can separate aerosols into nine distinct fractions based on aerodynamic size. The size cutoffs of each stage are dependent upon the flow rate at which the ACI is operated. Preferably the ACI is calibrated at 60 L/min. In one embodiment, a two-stage collapsed ACI is used for particle optimization. The two-stage collapsed ACI consists of stages 0, 2 and F of the eight-stage ACI and allows for the collection of two separate powder fractions. At each stage an aerosol stream passes through the nozzles and impinges upon the surface. Particles in the aerosol stream with a large enough inertia will impact upon the plate. Smaller particles that do not have enough inertia to impact on the plate will remain in the aerosol stream and be carried to the next stage.

The gravimetric fine particle fractions as a percentage of the total powder (FPF_{TP}<5.8 μm and FPF_{TP}<3.3 μm) were obtained gravimetrically at a flow rate of 28.3 L/min using stages 0, 1, and 3 of an Andersen Cascade Impactor (ACI) with effective cut-off diameters of 9.0, 5.8, and 3.3 μm, respectively. Filters were placed on the impaction plate below stage 3 and on the filter stage of the ACI. A flow meter, timing device, and vacuum pump were connected to the impactor and the flow rate was adjusted to 28.3 L/min. The inhaler was then actuated and powder was emitted, with a total volume of 2 L of air drawn through the inhaler and impactor. The difference in the filter weights before and after dose emission was used to calculate the gravimetric fine particle fractions.

The FPF of at least 45% of the particles of the invention is less than about 5.8 μm. For example, but not limited to, the FPF of at least 50%, or 60, or 70%, or 80%, or 90% of the particles is less than about 5.8 μm.

Another method for measuring the size distribution of airborne particles is the multi-stage liquid impinger (MSLI). The Multi-stage liquid Impinger (MSLI) operates on the same principles as the Anderson Cascade Impactor (ACI), but instead of eight stages there are five in the MSLI. Additionally, instead of each stage consisting of a solid plate, each MSLI stage consists of a methanol-wetted glass frit. The wetted stage is used to prevent bouncing and re-entrainment, which can occur using the ACI. The MSLI is used to provide an indication of the flow rate dependence of the powder. This can be accomplished by operating the MSLI at 30, 60, and 90 L/min and measuring the fraction of the powder collected on stage 1 and the collection filter. If the fractions on each stage remain relatively constant across the different flow rates then the powder is considered to be approaching flow rate independence.

The particles of the invention have a tap density of less than about 0.4 g/cm³. Particles which have a tap density of less than about 0.4 g/cm³ are referred to herein as “aerodynamically light particles.” For example, the particles have a tap density less than about 0.3 g/cm³, or a tap density less than about 0.2 g/cm³, a tap density less than about 0.1 g/cm³. Tap density can be measured by using instruments known to those skilled in the art such as the Dual Platform Microprocessor Controlled Tap Density Tester (Vankel, N.C.) or a GEOPYC™ instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10th Supplement, 4950-4951, 1999. Features which can contribute to low tap density include irregular surface texture and porous structure.

The envelope mass density of an isotropic particle is defined as the mass of the particle divided by the minimum sphere envelope volume within which it can be enclosed. In one embodiment of the invention, the particles have an envelope mass density of less than about 0.4 g/cm³.

The particles of the invention have a preferred size, e.g., a volume mean geometric diameter (VMGD) of at least about 1 micron. In one embodiment, the VMGD is from about 1 μm to 30 μm, or any subrange encompassed by about 1 μm to 30 μm, for example, but not limited to, from about 5 μm to about 30 μm, or from about 10 μm to 30 μm. For example, the particles have a VMGD ranging from about 1 μm to 10 μm, or from about 3 μm to 7 μm, or from about 5 μm to 15 μm or from about 9 μm to about 30 μm. The particles have a mean diameter, mass mean diameter (MMD), a mass median envelope diameter (MMED) or a mass median geometric diameter (MMGD) of at least 1 μm, for example, 5 μm or near to or greater than about 10 μm. For example, the particles have a MMGD greater than about 1 μm and ranging to about 30 μm, or any subrange encompassed by about 1 μm to 30 μm, for example, but not limited to, from about 5 μm to 30 μm or from about 10 μm to about 30 μm. A person skilled in the art can use the term “volume mean geometric diameter” and “volume median geometric diameter” interchangeably without regard to their statistical meaning.

The diameter of the spray-dried particles, for example, the VMGD, can be measured using a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition to targeted sites within the respiratory tract.

Aerodynamically light particles preferably have “mass median aerodynamic diameter” (MMAD), also referred to herein as “aerodynamic diameter”, between about 1 μm and about 5 μm or any subrange encompassed between about 1 μm and about 5 μm. For example, but not limited to, the MMAD is between about 1 μm and about 3 μm, or the MMAD is between about 3 μm and about 5 μm.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI).

The aerodynamic diameter, dₐₑᵣ, can be predicted from the equation:

dₐₑᵣ=d_{g} √ρₜₐₚ

where d_{g} is the geometric diameter, for example the MMGD, and ρ is the powder density.

Particles which have a tap density less than about 0.4 g/cm³, median diameters of at least about 1 μm, for example, at least about 5 μm, and an aerodynamic diameter of between about 1 μm and about 5 μm, preferably between about 1 μm and about 3 μm, are more capable of escaping inertial and gravitational deposition in the oropharyngeal region, and are targeted to the airways, particularly the deep lung. The use of larger, more porous particles is advantageous since they are able to aerosolize more efficiently than smaller, denser aerosol particles such as those currently used for inhalation therapies.

In comparison to smaller, relatively denser particles the larger aerodynamically light particles, preferably having a median diameter of at least about 5 μm, also can potentially more successfully avoid phagocytic engulfment by alveolar macrophages and clearance from the lungs, due to size exclusion of the particles from the phagocytes' cytosolic space. Phagocytosis of particles by alveolar macrophages diminishes precipitously as particle diameter increases beyond about 3 μm. Kawaguchi, H., et al., Biomaterials, 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). For particles of statistically isotropic shape, such as spheres with rough surfaces, the particle envelope volume is approximately equivalent to the volume of cytosolic space required within a macrophage for complete particle phagocytosis.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 μm are preferred for delivery to the central and upper airways. Particles having and aerodynamic diameter ranging from about 1 to about 3 μm are preferred for delivery to the deep lung.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. The aerodynamic diameter, daer, is related to the envelope sphere diameter, d (Gonda, I., “Physico-chemical principles in aerosol delivery,” in Topics in Pharmaceutical Sciences 1991 (eds. D. J. A. Crommelin and K. K. Midha), pp. 95-117, Stuttgart: Medpharm Scientific Publishers, 1992)), by the formula:

dₐₑᵣ=d√ρ

where the envelope mass ρ is in units of g/cm³. Maximal deposition of monodispersed aerosol particles in the alveolar region of the human lung (about 60%) occurs for an aerodynamic diameter of approximately dₐₑᵣ=3 μm Heyder, J. et al., J. Aerosol Sci., 17: 811-825 (1986). Due to their small envelope mass density, the actual diameter d of aerodynamically light particles comprising a monodisperse inhaled powder that will exhibit maximum deep-lung deposition is:

d=3/√ρ μm (where ρ 1 g/cm³);

where d is always greater than 3 μm. For example, aerodynamically light particles that display an envelope mass density, ρ 0.1 g/cm³, will exhibit a maximum deposition for particles having envelope diameters as large as 9.5 μm. The increased particle size diminishes interparticle adhesion forces. Visser, J., Powder Technology, 58: 1-10. Thus, large particle size increases efficiency of aerosolization to the deep lung for particles of low envelope mass density, in addition to contributing to lower phagocytic losses.

The aerodynamic diameter can be calculated to provide for maximum deposition within the lungs. Previously this was achieved by the use of very small particles of less than about five microns in diameter, preferably between about one and about three microns, which are then subject to phagocytosis. Selection of particles which have a larger diameter, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

In another embodiment of the invention, the particles have an envelope mass density, also referred to herein as “mass density” of less than about 0.4 g/cm³. Mass density and the relationship between mass density, mean diameter and aerodynamic diameter are discussed in U.S. Pat. No. 6,254,854, issued on Jul. 3, 2001, to Edwards, et al., which is incorporated herein by reference in its entirety.

Administration of particles to the respiratory system can be by means such as known in the art. For example, particles are delivered from an inhalation device such as a dry powder inhaler (DPI). Metered-dose-inhalers (MDI), nebulizers or instillation techniques also can be employed.

Various suitable devices and methods of inhalation which can be used to administer particles to a patient's respiratory tract are known in the art. For example, suitable inhalers are described in U.S. Pat. No. 4,069,819, issued Aug. 5, 1976 to Valentini, et al., U.S. Pat. No. 4,995,385 issued Feb. 26, 1991 to Valentini, et al., and U.S. Pat. No. 5,997,848 issued Dec. 7, 1999 to Patton, et al. Other examples include, but are not limited to, the SPINHALER®. (Fisons, Loughborough, U.K.), ROTAHALER®. (Glaxo-Wellcome, Research Triangle Technology Park, N.C.), FLOWCAPS®. (Hovione, Loures, Portugal), INHALATOR®. (Boehringer-Ingelheim, Germany), and the AEROLIZER®. (Novartis, Switzerland), the diskhaler (Glaxo-Wellcome, RTP, N.C.) and others, such as known to those skilled in the art. In one embodiment, the inhaler employed is described in U.S. Pat. No. 6,766,799, issued Jul. 27, 2004 to Edwards, et al., and in U.S. Pat. No 6,732,732, issued May 11, 2004 to Edwards, et al. The entire contents of these applications are incorporated by reference herein.

### Spray Drying

The invention also is related to producing particles that have compositions and aerodynamic properties described above. The method includes spray drying. Generally, spray-drying techniques are described, for example, by K. Masters in “Spray Drying Handbook”, John Wiley & Sons, New York, 1984.

The present invention is related to a method for preparing a dry powder composition. In this method, first and second components can be prepared, one of which comprises an active agent, a neuraminidase inhibitor, preferably, CS-8958. For example, the first component comprises an active agent e.g., a neuraminidase inhibitor dissolved in an organic solvent, and the second component comprises an excipient e.g., salt, buffer and amino acid, dissolved in an aqueous solvent. The first and second components can be combined either directly or through a static mixer to form a combination. The combination can be atomized to produce droplets that are dried to form dry particles. In one aspect of this method, the atomizing step can be performed immediately after the components are combined in the static mixer.

Suitable organic solvents that can be present in the mixture being spray dried include, but are not limited to, alcohols for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. In one embodiment, an ethanol/water solvent is preferred with the ethanol: water ratio ranging from about 30:70 to about 60:40. The mixture can have an acidic or alkaline pH. Preferably, the amount of organic solvent can be present in the co-solvent in an amount ranging from about 30 to about 90% by volume. In a more preferred embodiment, the organic solvent is present in the co-solvent in an amount ranging from about 45 to about 60% by volume. Optionally, a pH buffer can be included. Preferably, the pH can range from about 3 to about 10, for example, from about 6 to about 8.

An apparatus for preparing a dry powder composition is provided. The apparatus includes a static mixer (e.g., a static mixer as more fully described in U.S. Pat. No. 4,511,258, the entirety of which is incorporated herein by reference, or other suitable static mixers such as, but not limited to, model 1/4-21, made by Koflo Corporation) having an inlet end and an outlet end. The static mixer is operative to combine an aqueous component with an organic component to form a combination. Means are provided for transporting the aqueous component and the organic component to the inlet end of the static mixer. An atomizer is in fluid communication with the outlet end of the static mixer to atomize the combination into droplets. The droplets are dried in a dryer to form dry particles. The atomizer can be a rotary atomizer. Such a rotary atomizer may be vaneless, or may contain a plurality of vanes. Alternatively, the atomizer can be a two-fluid mixing nozzle. Such a two-fluid mixing nozzle may be an internal mixing nozzle or an external mixing nozzle. The means for transporting the aqueous and organic components can be two separate pumps, or a single pump. The aqueous and organic components are transported to the static mixer at substantially the same rate. The apparatus can also include a geometric particle sizer that determines a geometric diameter of the dry particles, and an aerodynamic particle sizer that determines an aerodynamic diameter of the dry particles.

The aqueous solvent and the organic solvent that make up the neuraminidase inhibitor solution are combined either directly or through a static mixer. The neuraminidase inhibitor solution is then transferred to the rotary atomizer (e.g., spray dryer) at a flow rate of about 5 to 28 g/min (mass) and about 6 to 80 ml/min (volumetric). For example, the neuraminidase inhibitor solution is transferred to the spray drier at a flow rate of 30 g/min and 31 ml/min. The 2-fluid nozzle disperses the liquid solution into a spray of fine droplets which come into contact with a heated drying air or heated drying gas (e.g., nitrogen) under the following conditions.

The pressure within the nozzle is from about 10 psi to 100 psi; the heated air or gas has a feed rate of about 80 to 110 kg/hr and an atomization flow rate of about 13 to 67 g/min (mass) and a liquid feed of 10 to 70 ml/min (volumetric); a gas to liquid ratio from about 1:3 to 6:1; an inlet temperature from about 90° C. to 150° C.; an outlet temperature from about 40° C. to 71° C.; a baghouse outlet temperature from about 42° C. to 55° C. For example, but not limited to, the pressure within the nozzle is set at 75 psi; the heated gas has a feed rate of 95 kg/hr; and an atomizer gas flow rate of 22.5 g/min and a liquid feed rate of 70 ml/min; the gas to liquid ratio is 1:3; the inlet temperature is 121° C.; the outlet temperature is 48° C.; the baghouse temperature is 43° C.

The contact between the heated nitrogen and the liquid droplets causes the liquid to evaporate and porous particles to result. The resulting gas-solid stream is fed to the product filter, which retains the fine solid particles and allows that hot gas stream, containing the drying gas, evaporated water and ethanol, to pass. The formulation and spray drying parameters are manipulated to obtain particles with desirable physical and chemical characteristics. Other spray-drying techniques are well known to those skilled in the art. An example of a suitable spray dryer using rotary atomization includes the Mobile Niro spray dryer, manufactured by Niro, Denmark. The hot gas can be, for example, air, nitrogen, carbon dioxide or argon.

The biocompatible particles of the invention are obtained by spray drying using an inlet temperature between about 90° C. and about 150° C. and an outlet temperature between about 40° C. and about 70° C.

The biocompatible particles can be fabricated with a rough surface texture to reduce particle agglomeration and improve flowability of the powder. The spray-dried particles have improved aerosolization properties. The spray-dried particle can be fabricated with features which enhance aerosolization via dry powder inhaler devices, and lead to lower deposition in the mouth, throat and inhaler device.

Methods and apparatus suitable for forming particles of the present invention are described in U.S. patent application Ser. No. 10/391,199 entitled “Method and Apparatus for Producing Dry Particles”, filed on Mar. 19, 2003 concurrently, which is a Continuation-in-part of U.S. patent application Ser. No. 10/101,563 entitled “Method and Apparatus for Producing Dry Particles”, filed on Mar. 20, 2002. The entire contents of these applications are incorporated by reference herein.

### EXAMPLES

### Experimental Procedures

A. General Methods

### Materials

Long-Acting Neuraminidase Inhibitor (LANI) compound CS-8958 was obtained from Sankyo Co.

### Production of AIR-LANI Powders by Spray-Drying

LANI powders were produced by spray drying solutions of dissolved raw materials. The drug, CS-8958, was dissolved in an organic solvent and the excipients were dissolved into either the aqueous or organic phase, where the organic solvent was typically ethanol, methanol, or an ethanol/water mixture. The solvent phases were separately pumped to a static mixer, where they were combined in the appropriate ratios by controlling the flow rates of the individual phases. The combined solution was pumped to either a two-fluid atomizer or a rotary atomizer in a size 1 Niro spray dryer.

The atomized liquid droplets were dried by heated nitrogen gas blown into the spray drying chamber. The dried powder then exited the spray dryer chamber and was carried to the product filter housing by the drying gas, where it was collected on a product filter bag. Powder was collected off the filter bag by pulsing with nitrogen, and using an air hammer on the filter housing to allow the powder to fall into the collection vessel at the bottom of the product filter housing. The collection vessel containing the powder was then removed from the system.

### Volume Mean Geometric Diameter (VMGD)

VMGD of bulk powders was determined using a HELOS diffractometer (Sympatec, Inc.) with a RODOS dispersion system operating at 1 bar. The HELOS diffractometer converts light scattering data into a geometric size distribution using an algorithm based on Fraunhofer diffraction.

### Gravimetric ACI-3 for Determination of FPF

The gravimetric fine particle fractions as a percentage of the total powder (FPF_{TP}<5.8 μm and FPF_{TP}<3.3 μm) were obtained gravimetrically at a flow rate of 28.3 L/min using stages 0, 1, and 3 of an Andersen Cascade Impactor (ACI) with effective cut-off diameters of 9.0, 5.8, and 3.3 μm, respectively. Filters were placed on the impaction plate below stage 3 and on the filter stage of the ACI. A flow meter, timing device, and vacuum pump were connected to the impactor and the flow rate was adjusted to 28.3 L/min. The inhaler was then actuated and powder was emitted, with a total volume of 2 L of air drawn through the inhaler and impactor. The difference in the filter weights before and after dose emission was used to calculate the gravimetric fine particle fractions. A flow rate of 28.3 LPM was used because the ACI was calibrated for this flow rate.

### Gravimetric Emitted Powder

The emitted powder was obtained gravimetrically by emission onto a filter contained in a sampling apparatus. A flow meter, timing device, and vacuum pump were connected to the sampling apparatus and the flow rate was adjusted accordingly. The inhaler was then actuated and flow was turned on for a total volume of 2 L. The difference in the filter weight before and after dose emission was used to calculate the gravimetric emitted powder.

### Short-Term Humidity Exposure

The short-term physical stability of AIR-LANI powders was tested by exposing them to various levels of humidity at ambient temperature for 24 hours, and then measuring the VMGD post-exposure to determine if there was any increase in size, indicating agglomeration of the particles. In order to expose samples of powder to various levels of humidity, open vials of bulk powder were placed in sealed chambers, in which the relative humidity of each chamber was controlled by enclosing a beaker containing a saturated solution of a salt. Saturated solutions of magnesium chloride, potassium carbonate, sodium bromide, and sodium chloride were used to generate approximately 33%, 42%, 57%, and 75% RH environments, respectively.

### Tapped Density

A known mass of powder was placed in a graduated container, which was placed in a Varian tap density instrument. Samples were tapped 500-1250 times per cycle until the volume change was <2% compared to the previous volume. Density was calculated as mass divided by final volume.

### Content

A RP-HPLC method developed by Sankyo Co. was used to assay drug content. Samples were prepared at a target concentration of 0.1 mg LANI/mL.

### Purity

Impurities were assessed using two gradient RP-HPLC methods developed by Sankyo Co. Samples were prepared at a target concentration of 1.0 mg LANI/mL for high drug loads such as the 30% CS-8958 formulation, and 0.5 mg/mL for low drug loads such as the 5% CS-8958 (LANI) formulation. All samples were prepared in duplicate.

### Water Content

Water content was determined using a Brinkmann (Metrohm) 756 Karl Fischer Coulometer with a 774 oven sample processor according to an Alkermes standard operating procedure.

### Example 1

### Production of 50% CS-8958 (LANI) Powders by Spray-Drying

The LANI compound CS-8958 was spray-dried with a number of different excipients, with the drug comprising 50% of the final composition. For the examples in Table 1, the spray-drying solutions, post-mixing, were made up of 60-80% Ethanol, and were atomized in a size 1 Niro spray-dryer using a two-fluid atomizer running at 12-45 g/min. atomization gas flow and 50-80 mL/min. total fluid flow rate.

TABLE 1
Spray-Dried Powders with 50% CS-8958 (LANI)

| Lot | Formulation | Formulation | VMGD | |
|---|---|---|---|---|
| No. | Ratio | Components | (μm) | Powder Handling |
| 1 | 50/45/5 | LANI/Leucine/ | 9 | poor; very static- |
| | | Sodium Phosphate | | sensitive |
| 2 | 50/50 | LANI/DPPC | 8 | static-sensitive |
| | | (phospholipid) | | |
| 3 | 50/30/15/5 | LANI/Leucine/ | 10 | poor; very static- |
| | | Trehalose/Sodium | | sensitive |
| | | Phosphate | | |
| 4 | 50/40/10 | LANI/DPPC/ | 14 | very static-sensitive |
| | | Sodium Citrate | | |
| 5 | 50/40/10 | LANI/DPPC/ | 16 | very static-sensitive |
| | | Sodium Chloride | | |
| 6 | 50/40/10/0.5 | LANI/DPPC/Citrate/ | 13 | very static-sensitive |
| | | Tween 80 | | |
| 7 | 50/25/20/5 | LANI/DPPC/Leucine/ | 14 | static-sensitive |
| | | Sodium Phosphate | | |
| 8 | 50/30/15/5 | LANI/DPPC/Mannitol/ | 24 | static-sensitive |
| | | Sodium Phosphate | | |
| 9 | 50/30/20 | LANI/DPPC/Arginine | 15 | very static-sensitive |
| 10 | 50/35/10/5 | LANI/Leucine/ | 9 | less static-sensitive, |
| | | Sodium Chloride/ | | more easily handled |
| | | Sodium Phosphate | | |

### Example 2

### Physical Stability Testing by Short-Term Humidity Exposure

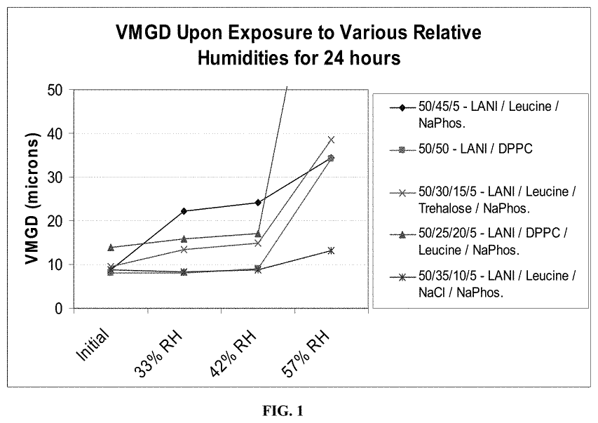

Selected formulations were exposed in bulk form to various levels of humidity at room temperature, and evaluated for changes in geometric size indicative of particle agglomeration, as detailed in the method for short-term humidity exposure (FIG. 1).

These studies clearly demonstrated significant differences between formulations, in terms of their physical stability under moderate stress conditions.

### Example 3

### Effect of Drug Load on Physical Properties of Spray-Dried Powders

Several of the excipient combinations in Example 1 were also spray-dried with varying amounts of LANI included in the composition. For the examples in Table 2, the spray-drying solutions, post-mixing, were made up of 60-80% Ethanol in water, and were atomized in a size 1 Niro spray-dryer using a two-fluid atomizer running at 12-30 g/min. atomization gas flow and 50-80 mL/min. total fluid flow rate.

TABLE 2
Spray-Dried Powders with 20-50% CS-8958 LANI

| Lot | Formulation | Formulation | VMGD | |
|---|---|---|---|---|
| No. | Ratio | Components | (μm) | Powder Handling |
| 7 | 50/25/20/5 | LANI/DPPC/Leucine/ | 14 | static-sensitive |
| | | Sodium Phosphate | | |
| 11 | 20/40/32/8 | LANI/DPPC/Leucine/ | 10 | minimal static |
| | | Sodium Phosphate | | sensitivity |
| 10 | 50/35/10/5 | LANI/Leucine/ | 9 | less static-sensitive, |
| | | Sodium Chloride/ | | more easily handled |
| | | Sodium Phosphate | | |
| 12 | 20/65/10/5 | LANI/Leucine/ | 5 | no static sensitivity, |
| | | Sodium Chloride/ | | very easy to handle |
| | | Sodium Phosphate | | |

These examples demonstrate the changes in size and powder handling that resulted from altering the load of CS-8958 (LANI) in the particles.

### Example 4

### Preparation and Characterization of Spray-Dried Powders Containing Leucine, Sodium Chloride, and Sodium Phosphate

Several batches of spray-dried powder were prepared using various ratios of leucine, sodium chloride, sodium phosphate, and CS-8958 (LANI), and using various process conditions including at least two methods of atomization. The examples in Table 3 were produced using a solution made up of 45-60% ethanol in water (v/v), atomized using either a two-fluid atomizer operating at 12-20 g/min. atomization gas flow, or a rotary atomizer operating at 20,000-50,000 rpm.

The powder batches listed in Table 3 were also characterized in terms of the tapped density of the bulk powder and the gravimetric fine particle fraction (FPF_{TP}<5.8 μm) of the powder when emitted out of an AIR inhaler with an airflow of 28.3 LPM.

TABLE 3
Spray-Dried Powders containing Leucine, Sodium Chloride, and
Sodium Phosphate with 5-40% CS-8958 (LANI)

| | Formulation Ratio | | | | |
|---|---|---|---|---|---|
| | (LANI/Leucine/ | | | Tapped | |
| Lot | Sodium Chloride/ | Atomizer | VMGD | Density | FPF_{TP} < |
| No. | Sodium Phosphate) | Type | (μm) | (g/cc) | 5.8 μm |
| 13 | 30/50/15/5 | Two-fluid | 6 | 0.12 | 56 |
| 14 | 30/50/15/5 | Rotary | 6 | 0.18 | 52 |
| 15 | 5/85/5/5 | Rotary | 5 | 0.23 | 50 |
| 16 | 40/45/10/5 | Rotary | 6 | 0.31 | 32 |

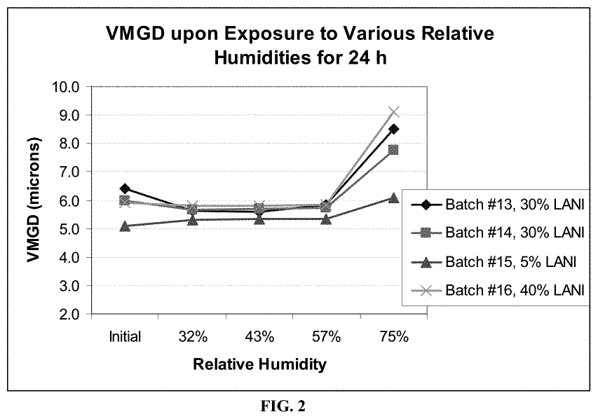

The above powders were also exposed in bulk form to various levels of humidity at room temperature, and evaluated for changes in geometric size indicative of particle agglomeration, as detailed in the method for short-term humidity exposure (FIG. 2).

These powders, which represent a range of drug loads and process conditions, demonstrate physical stability up through the moderately severe stress condition of 57% relative humidity.

### Example 5

### One-Month Stability of AIR-LANI Powders

Two formulations, equivalent to batches 14 (30% CS-8958 (LANI)) and 15 (5% CS-8958 (LANI)) above, were selected for manufacture at a larger scale, and evaluated into a one-month stability study. The powders were packaged into HPMC capsules in blister packs, and sealed in foil pouches. Stability conditions were 25° C./60% RH, as a likely storage condition, and 40° C./75% RH as an accelerated condition. After storage at each stability condition, the powders were evaluated in terms of gravimetric emitted powder, gravimetric fine particle fraction, content, purity, and water content. The results for the 30% CS-8958 (LANI) powder are summarized in Table 4, and the results for the 5% CS-8958 (LANI) powder are summarized in Table 5. These data are indicative of robust formulations with good storage stability.

TABLE 4
One-Month Stability Summary for 30% CS-8958 (LANI) Powder

| | | | Storage | | Storage | |
|---|---|---|---|---|---|---|
| | | | Condition: | | Condition: | |
| | | | 25° C./ | | 40° C./ | |
| | | | 60% RH | | 75% RH | |
| Method | Initial | | 4 Weeks | | 4 Weeks | |
| % Gravimetric Emitted Powder | 82 | (5) | 87 | (2) | 87 | (1) |
| Mean (SD) | | | | | | |
| % Gravimetric Fine Particle | 47 | (1) | 51 | (9) | 53 | (3) |
| Fraction (FPF_{TP} < 5.8 μm) | | | | | | |
| Mean (SD) | | | | | | |
| % Gravimetric Fine Particle | 19 | (3) | 17 | (2) | 19 | (1) |
| Fraction (FPF_{TP} <3.3 μm) | | | | | | |
| Mean (SD) | | | | | | |
| % Content | 30.4 | (0.5) | 29.2 | (0.1) | 29.5 | (0.1) |
| Mean (SD) | | | | | | |
| % Impurities Sankyo Method 1 | 0.29 | | 0.31 | | 0.27 | |
| Mean | | | | | | |
| % Impurities Sankyo Method 2 | 0.00 | | 0.00 | | 0.07 | |
| Mean | | | | | | |
| % Water Content | 3.24 | (0.03) | 3.06 | (0.04) | 2.80 | (0.03) |
| Mean (SD) | | | | | | |

TABLE 5
One-Month Stability Summary for 5% CS-8958 (LANI) Powder

| | | | Storage | | Storage | |
|---|---|---|---|---|---|---|
| | | | Condition: | | Condition: | |
| | | | 25° C./ | | 40° C./ | |
| | | | 60% RH | | 75% RH | |
| Method | Initial | | 4 Weeks | | 4 Weeks | |
| % Gravimetric Emitted Powder | 77 | (9) | 82 | (2) | 83 | (2) |
| Mean (SD) | | | | | | |
| % Gravimetric Fine Particle | | | | | | |
| Fraction (FPF_{TP} < 5.8 μm) | 65 | (2) | 58 | (2) | 62 | (5) |
| Mean (SD) | | | | | | |
| % Gravimetric Fine Particle | | | | | | |
| Fraction (FPF_{TP} < 3.3 μm) | 35 | (1) | 30 | (1) | 30 | (2) |
| Mean (SD) | | | | | | |
| % Content | 4.90 | (0.01) | 4.92 | (0.03) | 4.83 | (0.01) |
| Mean (SD) | | | | | | |
| % Impurities Sankyo Method 1 | 0.22 | | 0.13 | | 0.08 | |
| Mean | | | | | | |
| % Impurities Sankyo Method 2 | 0.12 | | 0.05 | | 0.05 | |
| Mean | | | | | | |
| % Water Content | 1.48 | (0.03) | 1.54 | (0.02) | 1.54 | (0.03) |
| Mean (SD) | | | | | | |

### Example 6

### Open Stress Stability Study

In addition to the one-month storage stability study, the same two lots of CS-8958 (LANI) powders packaged in capsules were placed in a two-week open stress stability study. In this study, 20 mg of each formulation was placed in size 2 capsules and the “bare” capsules were directly exposed to the environments of the storage chambers. The storage conditions evaluated included 25° C./30% RH, 25° C./60% RH and 40° C./75% RH, and were controlled to within ±2-3° C. and ±5% RH. The product attributes studied include emitted powder assessment for dose delivery, gravimetric fine particle fraction (FPF_{TP}<5.8 μm), CS-8958 (LANI) content, purity and water content. The results for the 30% CS-8958 (LANI) powder at the 30% and 60% RH conditions are summarized in Table 6, and the results for the 5% CS-8958 (LANI) powder at the same two conditions are summarized in Table 7. At the 40° C./75% RH condition, both powders absorbed significantly more water and decreased fine particle fraction, consistent with the size change observed in the 24-hour exposure to 75% RH.

TABLE 6
Two-Week Open Stress Stability Summary for
30% CS-8958 (LANI) Powder

| | | | Storage | | Storage | |
|---|---|---|---|---|---|---|
| | | | Condition: | | Condition: | |
| | | | 25° C./ | | 25° C./ | |
| | | | 30% RB | | 60% RB | |
| Method | Initial | | 2 Weeks | | 2 Weeks | |
| % Gravimetric Emitted Powder | 82 | (5) | 84 | (5) | 89 | (2) |
| Mean (SD) | | | | | | |
| % Gravimetric Fine Particle | 47 | (1) | 52 | (2) | 57 | (1) |
| Fraction (FPF_{TP} < 5.8 μm) | | | | | | |
| Mean (SD) | | | | | | |
| % Gravimetric Fine Particle | 19 | (3) | 17 | (1) | 17 | (1) |
| Fraction (FPF_{TP} < 3.3 μm) | | | | | | |
| Mean (SD) | | | | | | |
| % Content, Mean (SD) | 30.4 | (0.5) | 28.1 | (0.2) | 27.5 | (0.2) |
| % Impurities Sankyo Method | 0.29 | | 0.32 | | 0.31 | |
| 1, Mean | | | | | | |
| % Impurities Sankyo Method | 0.00 | | 0.09 | | 0.05 | |
| 2, Mean | | | | | | |
| % Water Content, Mean (SD) | 3.24 | (0.03) | 2.96 | (0.06) | 5.71 | (0.05) |

TABLE 7
Two-Week Open Stress Stability Summary for
5% CS-8958 (LANI) Powder

| | | | Storage | | Storage | |
|---|---|---|---|---|---|---|
| | | | Condition: | | Condition: | |
| | | | 25° C./ | | 25° C./ | |
| | | | 30% RB | | 60% RB | |
| Method | Initial | | 2 Weeks | | 2 Weeks | |
| % Gravimetric Emitted Powder | 77 | (9) | 82 | (2) | 86 | (3) |
| Mean (SD) | | | | | | |
| % Gravimetric Fine Particle | 65 | (2) | 60 | (2) | 59 | (3) |
| Fraction (FPF_{TP} < 5.8 μm) | | | | | | |
| Mean (SD) | | | | | | |
| % Gravimetric Fine Particle | 35 | (1) | 31 | (1) | 30 | (2) |
| Fraction (FPF_{TP} < 3.3 μm) | | | | | | |
| Mean (SD) | | | | | | |
| % Content, Mean (SD) | 4.90 | (0.01) | 4.75 | (0.02) | 4.62 | (0.01) |
| % Impurities Sankyo Method | 0.22 | | 0.27 | | 0.23 | |
| 1, Mean | | | | | | |
| % Impurities Sankyo Method | 0.12 | | 0.11 | | 0.10 | |
| 2, Mean | | | | | | |
| % Water Content, Mean (SD) | 1.48 | (0.01) | 1.33 | (0.01) | 3.40 | (0.03) |

### REFERENCES

K. S. Li, Nature 430, 209-213 (8 Jul. 2004) Genesis of a highly pathogenic and potentially pandemic H5N1 influenza virus in eastern Asia.

What is claimed is:

1. A mass of biocompatible particles suitable for delivery to the pulmonary system consisting essentially of, by weight, about 5% to about 50% of a neuraminidase inhibitor, sodium chloride, a buffer and, an amino acid wherein the particles are free of phospholipid and have a fine particle fraction of less than 5.8 μm of at least 45% by weight.

2. A mass of biocompatible particles suitable for delivery to the pulmonary system consisting of, by weight, about 5% to about 50% of a neuraminidase inhibitor, sodium chloride, a buffer and, an amino acid wherein the particles have a fine particle fraction of less than 5.8 μm of at least 45% by weight.

3. The mass of biocompatible particles of claim 2, wherein the amino acid is leucine and the buffer is sodium phosphate.

4. The mass of biocompatible particles of claim 2, wherein the particles have a tap density of less than or about 0.1 g/cm³.

5. The mass of biocompatible particles of claim 2, wherein the particles have a median geometric diameter of from about 5 micrometers to about 30 micrometers.

6. The mass of biocompatible particles of claim 2, wherein the particles have an aerodynamic diameter from about 1 micrometer to about 3 micrometers.

7. The mass of biocompatible particles of claim 2, wherein the particles are spray-dried.

8. A pharmaceutical formulation having particles consisting essentially of, by weight, about 5% to about 30% of a neuraminidase inhibitor, about 5% to about 20% sodium chloride, about 20% to about 85% leucine and about 5% to about 20% sodium phosphate wherein the particles are free of phospholipid and have a fine particle fraction of less than 5.8 μm of at least 45% by weight.

9. A pharmaceutical formulation having particles consisting of, by weight, about 5% to about 30% of a neuraminidase inhibitor, about 5% to about 20% sodium chloride, about 20% to about 85% leucine and about 5% to about 20% sodium phosphate wherein the particles have a fine particle fraction of less than 5.8 μm of at least 45% by weight and have a fine particle fraction of less than 5.8 μm of at least 45% by weight.

10. The pharmaceutical formulation of claim 9, wherein the particles consist of 30% of a neuraminidase inhibitor, 15% sodium chloride, 50% leucine and 5% sodium phosphate.

11. The pharmaceutical formulation of claim 9, wherein the particles consist essentially of 5% of a neuraminidase inhibitor, 5% sodium chloride, 85% leucine and 5% sodium phosphate.

12. A method of treating a human subject in need of a neuraminidase inhibitor comprising administering pulmonarily to the respiratory tract of a subject in need of treatment an effective amount of a mass of particles according to claim 1, wherein the release of the neuraminidase inhibitor is rapid.

13. The method of claim 12, wherein the subject in need of treatment has influenza.
